(19) United States
(12) Patent Application Publication
(10) Pub. No.: US 2016/0367968 A1
(43) Pub. Date: Dec. 22, 2016

(54) Photocatalysts

(71) Applicant: QUEEN MARY UNIVERSITY OF LONDON, London (GB)

(72) Inventors: Igor Larrosa Guerrero, London (GB); Steven Colin Dunn, London (GB); Armando Marsden Lacerda Neto, London (GB)

(73) Assignee: Queen Mary University of London, London (GB)

(21) Appl. No.: 15/031,738

(22) PCT Filed: Oct. 24, 2014
(86) PCT No.: PCT/GB14/53191

§ 371 Date: Apr. 24, 2016

(30) Foreign Application Priority Data
Oct. 24, 2013 (GB) 1318846.1

(51) Int. Cl. B01J 23/44; B01J 21/06; B01J 37/34; B01D 53/00; C01B 3/04; A61L 9/18; C02F 1/30; B01J 35/00; B01J 37/06

(52) U.S. Cl. CPC B01J 23/44; B01J 35/004; B01J 21/063; B01J 37/345; B01J 37/06; B01J 35/0013; C01B 3/042; A61L 9/18; C02F 1/30; B01D 53/007; A61L 2209/12; C02F 2303/04; C02F 2305/08; C02F 2305/10; B01D 2255/802; B01D 2259/802

(57) ABSTRACT

The present invention provides photocatalysts capable of catalytic activity in the visible range of light comprising platinum group metal nanoparticles deposited on a metal oxide support. The nanoparticles have surface plasmon resonance in the visible range of light. The invention also provides processes for preparing the photocatalysts, methods of liquid and gas purification using the photocatalysts of the invention and devices for the same.

The present invention relates to novel photocatalysts and uses thereof. The invention also relates to processes for preparing the novel photocatalysts.

Fresh water is our planet's most valuable resource accounting for less than 10% of all available water on the surface. WHO estimates that 10% of the health burden can be relieved by improving water quality. Poor water quality is especially a problem in developing countries where studies suggest that up to 90% of wastewater flows untreated into rivers, lakes and coastal zones. It is estimated that polluted water affects the health of more than 1.2 billion people and contributes to the death of approximately 15 million children every year. Contamination of water by organic compounds is a growing concern all over the world. Many organic compounds can mimic hormones and have an effect on people at very low concentrations. Others have been linked to different cancers. Organic pollution also affects and can potentially destroy aquatic ecosystems. Common sources of organic pollutants include industrial effluents for example from chemical, textile and leather industries, agricultural wastewater and domestic sewage.

Titanium dioxide (TiO₂) is widely used as a photocatalyst in water purification systems. It is a cheap, naturally occurring, commonly available oxide of titanium and has a good safety profile. A major drawback of TiO₂ is that high energy light such as ultraviolet (UV) light is necessary to activate it, necessitating the use of an artificial, and usually expensive, UV source in the purification system. UV light constitutes approximately 2-4% of sunlight. The efficiency of TiO₂ is therefore limited by its ability to absorb only a small fraction of the available light.

Papp et al. (Chem. Mater. 1993, 5, 284-288) disclose that addition of palladium to TiO₂ increases its photocatalytic activity. However, UV light is still needed to activate the TiO₂.

There is therefore a need for a more efficient photocatalyst that can show catalytic activity in the visible range of light.

The present invention provides novel photocatalysts having improved photocatalytic activity in visible light.

The present invention provides photocatalysts capable of catalytic activity in the visible range of light comprising platinum group metal nanoparticles deposited on a metal oxide support. The nanoparticles have surface plasmon resonance in the visible range of light. The invention also provides processes for preparing the photocatalysts, methods of liquid and gas purification using the photocatalysts of the invention and devices for the same.

In a first aspect of the invention there is provided a photocatalyst comprising platinum group metal nanoparticles on a metal oxide support. The nanoparticles have surface plasmon resonance in the visible range of light. The photocatalysts are capable of photocatalytic activity in the visible range of light. The nanoparticles are deposited on the metal oxide and are amorphous.

As used herein, “photocatalyst” refers to a substance that increases the rate of a chemical reaction requiring the presence of light. The catalytic activity of a photocatalyst depends on its ability to generate electron-hole pairs which then participate in and accelerate downstream reactions. As used herein, “visible range of light” refers to the range of light visible to the naked human eye. Generally, the visible range of light is electromagnetic radiation with wavelength greater than or equal to about 380 nm, 390 nm, 400 nm, 410 nm, 420 nm, 430 nm, 440 nm or 450 nm, or up to about 650 nm, 660 nm, 670 nm, 680 nm, 690 nm, 700 nm, 710 nm, 720 nm for example between about 390 nm and about 700 nm.

Platinum group metals include ruthenium, rhodium, palladium, osmium, iridium, and platinum. In an embodiment of the invention the platinum group metal is palladium or platinum. In some embodiments, the platinum group metal is palladium.

Metal oxides (or other compounds for use in combination with the platinum group metal) used in the invention include, but are not limited to, titanium dioxide (TiO₂), zinc oxide (ZnO), cadmium sulfide (CdS), barium titanate (BaTiO₃), zirconium dioxide (ZrO2), tungsten oxide (WO₃), potassium niobate crystal (KNbO₃), or strontium titanate (SrTO₃).

In an embodiment of the invention the metal oxide is a refractory metal oxide. Refractory metals include titanium, chromium, zirconium, niobium, molybdenum, hafnium and tungsten.

In a preferred embodiment of the invention the metal oxide is a titanium oxide, such as titanium dioxide (TiO₂).

TiO₂ has three main crystalline structures: anatase, rutile and brookite. Degussa P-25 is a standard material in the field of photocatalytic reactions containing anatase and rutile phases in a ratio of about 3:1. The photocatalysts of the invention comprising TiO₂ may include anatase, rutile or brookite crystalline structures, or a combination thereof. For example, the photocatalysts of the invention comprising TiO₂ may include a combination of anatase and rutile phases, for example in a ratio of about 3:1. In some embodiments, the photocatalysts do not contain the brookite phase of TiO₂.

In one embodiment the TiO₂ (or other metal oxide) is in a powdered form with an average particle size between about 20 and about 25 nm, such as Degussa P-25 (CAS No. 13463-67-7, commercially available from Evonik). The TiO₂ (or other metal oxide), when in powdered form, may have a surface specific area (BET) of between about 30 and about 70 m²/g, for example between about 35 and about 65 m²/g. The tapped density (according to DIN EN ISO 789/11, August 1983) may be about 100 to about 150 g/L, for example between about 120 and about 140 g/L. The TiO₂ (or other metal oxide) may have a combination of these features, for example an average particle size of between about 20 and about 25 nm, a surface specific area of about 35 to about 65 m²/g, and optionally a tapped density of between about 120 and about 140 g/L. The photocatalyst may maintain some or all of these properties when formed from such metal oxides.

Generally, the metal oxides are in a powder form (such as a crystalline form), for example with an average particle diameter of up to about 50 nm, optionally up to about 40 nm or up to about 30 nm. In some embodiments, the average particle diameter is more than about 10 nm, for example more than about 20 nm. The average particle diameter may be between about 10 and about 50 nm, for example, between about 20 and about 30 nm, between about 20 and about 25 nm and most preferably about 25 nm. Alternatively, the metal oxides may be in solution, such as an aqueous solution, for example between 1 and 10 g/L, or between 1 and 5 g/L, optionally 2 g/L. The solutions may be made using the powdered metal oxides above. Similarly, the photocatalysts of the invention may be present in a powdered (such as crystalline) form or in solution, such as in water, optionally deionised water, or in suspension. The physical properties of the photocatalysts may be as provided above for the metal oxides.

As used herein, “nanoparticle” refers to any particle having a diameter of less than about 1000 nanometers (nm).

In an embodiment of the invention the nanoparticles are deposited on a metal oxide, in particular on the surface of the metal oxide. The platinum metal can be considered a co-catalyst.

In another embodiment of the invention the platinum group metal nanoparticles are deposited on a metal oxide support. In some embodiments, the nanoparticles are amorphous. In particular embodiments, the nanoparticles are not in a crystalline form. Generally, the atomic percentage of photo-deposited metal to metal catalyst is about 0.4%, for example between about 0.3 and about 0.5%. In some embodiments, the atomic percentage of photo-deposited metal to metal catalyst is up to 1%, optionally up to 7%, up to 5% or up to 4%. In the case of a palladium-supported TiO₂ photocatalyst, there may be an average of about 4 mg per gram of palladium to titanium dioxide or about 5 mg per gram of palladium to titanium dioxide. In some embodiments, there is up to about 10 mg of platinum group metal per gram of metal oxide, for example, up to about 9 mg, up to about 8 mg, up to about 6 mg, up to about 5 mg or up to about 4 mg platinum group metal to gram of metal oxide. In some embodiments there is at least about 2 mg, about 3 mg, about 4 mg, about 5 mg or about 6 mg platinum group metal per gram of metal oxide. In some embodiments, there is about 3 to 7, about 4 to about 6 or about 5 mg of platinum group metal per g of metal oxide in the photocatalysts of the invention. In one embodiment of the invention, there is about 4.8 to about 6.3 mg of platinum group metal (such as palladium) per gram of metal oxide (such as TiO₂).

The metal oxide can also be doped to make it a better catalyst. Doping is known in the art and refers to the process of intentionally introducing impurities into a substance to enhance the substance's charge carrier density. Doping of the metal oxide generally occurs during the manufacture of the metal oxide prior to the manufacture of the photocatalyst. Doping may be achieved using, for example, nitrogen as the impurity. Other impurities may be incorporated, for example platinum or noble group metals may be used as dopants. Dopants are generally incorporated during the synthesis procedure of the metal oxide (for example a titanium metal oxide such as TiO₂). The dopant ions usually replace an ion in the metal oxide lattice, and so form part of the metal oxide support that later has the nanoparticles deposited onto it. This can be done using, for example, a hydrothermal synthesis procedure of the catalyst. The amount of dopant present will depend on the concentration of the dopant solution and other parameters of the synthesis such as temperature and time.

In some embodiments, therefore, the photocatalyst of the invention may further comprise an impurity, specifically a deliberate impurity (dopant). In other embodiments of the invention, however, the metal oxide is not doped.

The catalytic activity of TiO₂ in the presence of light has been studied intensively and is widely used for example in water purification, hydrogen production and, antifogging coatings. TiO₂ can be used in water purification. Photocatalysts of the present invention can be used in such applications as well.

The energy gap between the valence and conduction bands in TiO₂ is approximately 3-3.2eV. Due to this large band gap, activation of TiO₂ is usually restricted to high energy light, i.e. ultra violet light (UV). In order to use visible light to activate TiO₂, this band gap needs to be reduced.

Upon activation by light, valence band electrons in TiO₂ are excited to the conduction band resulting in the formation of electron-hole pairs which diffuse to the surface of the TiO₂. The electron in the conduction band participates in reduction reactions whereas the hole in the valence band takes part in oxidation reactions, each leading to the production of reactive species. For example, when placed in water, the electron combines with the oxygen in the water to form a reactive oxygen species such as a superoxide anion or a peroxide and the hole leads to the splitting of water into a hydroxyl radical and a proton. The reactive oxygen species and hydroxyl radical are highly reactive and interact with organic compounds in the water thus degrading them.

The reactive species can also interact with the cell membranes of microorganisms leading to lysis of the microorganism.

In some embodiments of the invention the photocatalyst is antimicrobial. There is therefore provided the use of the photocatalysts of the invention as antimicrobial agents. There is also provided a method of sterilising, purifying or decontaminating a liquid or gas, comprising mixing a liquid or gas with a photocatalyst of the invention and applying visible light to the resulting mixture. The light activates the photocatalyst and the liquid or gas is sterilised. The photocatalyst may be added to the liquid or gas as a solid (for example a powder) or as a liquid (for example in aqueous solution). In methods of the invention (methods of sterilisation, purification or decontamination), the photocatalyst may optionally be removed after sterilisation/purification/decontamination.

Surface plasmon resonance (SPR) refers to the collective resonance or oscillation of free electrons at the interface of a solid or liquid and a dielectric in response to excitation by incident light when the frequency of the incident light matches the natural frequency of the electrons. A plasmon is a quantum of collective oscillation of free electrons. It also refers to an electromagnetic wave formed as a result of the collective oscillation.

Palladium particles show plasmons in the UV range. However, the inventors have found that palladium nanoparticles with particle size between, for example, about 2 nm to about 5 nm show plasmons in the visible range.

In a preferred embodiment of the invention, the platinum group metal nanoparticle is a palladium nanoparticle.

In an embodiment of the invention the platinum group metal (such as palladium) nanoparticle has a size (diameter) up to about lOnm, about 8 nm, about 6 nm or preferably up to about 5 nm. In an embodiment of the invention the platinum group metal (such as palladium) nanoparticle has a size of at least about lnm, about 2 nm, about 3 nm or up to about 4 nm. In a preferred embodiment of the invention the nanoparticles have an average size (diameter) between about lnm and about 10 nm, about lnm and about 8 nm, about 2 nm and about 8 nm, about 2 nm and about 7 nm, about 2 nm and about 6 nm, or about 2 nm and about 5 nm.

Nanoparticles can be deposited onto the metal oxide (such as TiO₂) via a photocatalytic mechanism or from nanoparticle formation in solution followed by adsorption onto the surface. In some embodiments of the invention the nanoparticles are deposited by UV photodeposition.

UV photodeposition can be carried out for up to about 30 minutes, for example about 25 min, about 20 min, about 15 min, about 10 min, about 5 min, about 1 min, about 30 seconds, about 15 seconds, about 10 seconds, about 5 seconds or about 1 second. Generally speaking, a platinum group metal salt solution, for example at a concentration of up to 0.02 mol/L, is mixed with the metal oxide (for example up to 1 gram of the metal oxide such as TiO₂ (P25)). Optionally this can be done a glass dish fitted with a quartz lid. The solution may be stirred under UV irradiation. The resulting photocatalysts may be extracted from the solution, for example by drying.

Rhodamine B is an organic compound that is commonly used as a dye. The photocatalysts of the invention can be tested for photocatalytic activity by measuring dye (such as Rhodamine B) degradation. The photocatalysts of the invention can be tested for catalytic activity by measuring the degradations of other compounds such as chlorobenzene compounds, sodium dodecylbenzenesulphonate (DBS) or benzoic acids. Dyes other than Rhodamine B include methyl orange and methylene blue. Degradation of dyes can be measured by decolourisation (for example using a colorimeter). Degradation of other compounds can be measured by, for example, gas chromatography. Alternatively, the total organic content (total amount of carbon at the beginning and at different points during the reaction process over time) can be measured. A standard reaction for measuring the photocatalytic activity of a test compound (such as TiO₂) is typically the measurement of the decrease in concentration of a pollutant introduced to an aqueous solution in the presence of an irradiation source to activate the catalyst. The pollutant may be a compound that degrades on activation of the photocatalyst, such as a dye (for example Rhodamine B, methyl orange and methylene blue), or other compound such as chlorobenzene compounds, sodium dodecylbenzenesulphonate (DBS) or benzoic acids.

Generally, the photocatalysts of the invention will catalyse a reaction (for example the degradation of Rhodamine B) by up to about 5-fold, for example up to about 10-fold, up to about 15-fold, up to about 20-fold, up to about 25-fold or up to about 30-fold. The photocatalysts of the invention may catalyse such a reaction by at least about 10-fold or by at least about 15-fold or by at least about 20-fold or by at least about 25-fold. In some embodiments, the photocatalysts catalyse reactions, such as the degradation of Rhodamine B, by between about 5 and about 30-fold, for example between about 10 and about 30-fold or between about 15 and about 30-fold.

In a second aspect of the invention there is provided a purification device comprising a photocatalyst according to the first aspect of the invention. The device may be a liquid (eg water) or gas (eg air) purification device. Sterilisation and decontamination devices are also provided, and these have the same features as the described purification devices.

A purification device as provided herein generally refers to a liquid purification system or a gas purification system. In an embodiment of the invention, the liquid purification system is a water purification system.

TiO₂ is very commonly used in water purification systems. A water purification system typically comprises a polluted water inlet, a purification chamber and a treated water outlet. The purification chamber of the prior art comprises TiO₂ and a UV light source. Polluted water enters the system through the inlet and interacts with the TiO₂, which is activated by the UV light resulting in the formation of reactive species. Organic compounds and microorganisms in the water are degraded by the reactive species and the purified water exits the system through the outlet. The purification chamber may also act as a storage chamber, or alternatively there may be a storage chamber in fluid communication with the purification chamber via the water outlet where purified water is stored until it is required. The storage chamber may itself have a further water outlet allowing the purified water to be dispensed from the purification device.

The TiO₂ in a water purification system, such as of the type described above, can be replaced with the photocatalyst of the invention and hence in embodiments of the invention the water purification system includes a photocatalyst of the invention in the purification chamber. Thus visible light can be used to activate the catalyst and purify the water. However, UV light can still be used since the catalysts of the invention are capable of catalysis in the UV spectrum (for example between 10 and 400 nm or between 10 and 390 nm) as well as in the visible light spectrum.

In an embodiment of the invention the gas purification system is an air purification system.

The purification devices of the invention comprise a reaction chamber having an inlet and an outlet. The reaction chamber comprises the photocatalyst of the invention and this is where the purification takes place. Up to about lg, up to about 500mg, up to about 100 mg or up to about 50mg of photocatalyst may be present. In some embodiments, at least about 10mg, at least about 50mg, at least about 100mg or at least about 500mg of photocatalyst may be present. In the case of liquid purification systems, the photocatalyst may be present in solution or suspension. In the case of gas purification systems, the photocatalyst may be present as a bed of solid or powdered catalyst through or over which the gas to be purified flows.

The inlet is an inlet for the liquid or gas to be purified. In some embodiments, for example in the case of a liquid purification device, the inlet may simply be a removable lid of the reaction chamber, although in other embodiments the inlet may be a hollow conduit (such as a pipe). The outlet is for purified liquid or gas, and similarly may be a hollow conduit (such as a pipe). The inlet may comprise a filter for removing particulate contaminants. The outlet pipe may comprise means for removing the photocatalyst from the purified liquid or gas, such as a filter.

Alternatively, the means for removing the catalyst may be a centrifuge or a means for distillation that is in fluid communication with the reaction chamber via the reaction chamber outlet.

The purification device may optionally include a source of light, such as a source of visible light. The reaction chamber may be transparent, for example if the source of light located externally to the reaction chamber. Alternatively, the source of light may be located inside the reaction chamber. The source of light may be operably linked to a control means that allows a user to activate or deactivate the source of light.

The purification device may further comprise a storage chamber to store purified liquid or gas. The storage chamber, if present, is in fluid communication with the reaction chamber via the reaction chamber outlet. The storage chamber may further comprise a dispensing outlet having a valve.

The storage chamber may itself be connected to a means for removing the photocatalyst described herein, for example via its dispensing outlet. Alternatively, the means for removing the photocatalyst described herein may comprise a chamber in fluid communication with the reaction chamber via the reaction chamber outlet. The chamber of the means for removing the photocatalyst may then be in further fluid communication with the storage chamber via a storage chamber inlet. The storage chamber is therefore useful for storing purified liquid or gas from which the photocatalyst has been removed.

Pumps may also be present. For example, there may be a pump for feeding gas or liquid into the reaction chamber via the inlet and/or a pump for expelling purified gas or liquid from the reaction chamber via the outlet (optionally into the storage chamber, if present). If a storage chamber with dispensing outlet is present, the flow of liquid or gas through the dispending outlet may be effected by means of a pump (optionally operably linked to a control means).

Generally, the inlets and outlets will comprise valves for controlling the flow of water through them. Control means may be present that are operably linked to the valves so a user can control the flow of liquid or gas. In particular, the purification device may comprise a control means that is operably linked to the valve of the reaction chamber outlet (or the valve of the storage chamber dispensing outlet) allowing purified liquid or gas to be dispensed. The control means may also be operably linked to any pumps present.

The purification device may include a storage chamber and further a feedback loop for recirculating the liquid or gas multiple times. The feedback loop allows the liquid or gas to exit and then re-enter the reaction chamber. In such embodiments, the feedback loop comprises a valve that determines the flow of the liquid or gas either through the reaction chamber outlet into the storage chamber (once the liquid or gas is suitably purified) or back into the reaction chamber via a conduit to permit further purification. The purification device may include means for testing the level of purification of the gas or liquid. This allows a user to determine when a suitable amount of purification has taken place, or this may be done automatically by the system itself. Optionally, the means for testing the level of purification in the liquid or gas is located in the feedback loop and is operably linked to the valve therein, such that the system automatically recirculates polluted liquid or gas until a desired level of purification has taken place.

In a third aspect of the invention there is provided a hydrogen production device.

The apparatus for the production of hydrogen from water or aqueous solutions of organic compounds by using the catalyst comprises a light source (such as a visible light source), a reactor (optionally wherein the reactor is transparent for the light of the light source if the light source is external to the reactor), an inlet for feeding water or aqueous solution to the reactor, and a gas product outlet for releasing hydrogen liberated in the reaction chamber. The photocatalyst of the invention is present in the reactor. The apparatus for the production of hydrogen may further comprise a storage chamber for collecting and storing the hydrogen produced. The storage chamber is in communication with the reaction chamber via the gas outlet. The storage chamber may be pressurised.

Valves may also be present, to control the flow of water or aqueous solution into the reactor via the inlet and release of gas via the outlet. Control means also be present to adjust the light source intensity or even switch it on or off as required. The reaction chamber may further comprises a waste outlet for removal of waste or by-products or unreacted water or aqueous solution, the waste outlet optionally having a valve. Still further, the hydrogen production device may comprise control means operably linked to the valves for controlling the flow water or aqueous solution into the reaction chamber, the flow of hydrogen through the outlet (and into the storage chamber if present), and/or the flow of waste or by-products or unreacted water or aqueous solution through the waste outlet.

In devices of the invention (purification, decontamination, sterilisation or hydrogen production devices), the photocatalyst of the invention may be present in the reaction chamber as a solid (eg a powder or in crystalline form), or alternatively it may be present in solution, such as in an aqueous solution, or suspension. The devices may further comprise a means for adding the photocatalyst to the reaction chamber (or for replenishing the photocatalyst), for example a photocatalyst inlet in communication with the reaction chamber. The means for adding the photocatalyst to the reaction chamber may be a removable lid of the reaction chamber. Such a lid would also facilitate cleaning and maintenance.

In a fourth aspect of the invention there is provided a process for preparing a photocatalyst of the invention. The process comprises depositing a platinum group metal (such as palladium) on a metal oxide (for example an oxide of a refractory metal, such as a titanium oxide). The platinum group metal is deposited in the form of nanoparticles. The nanoparticles have a surface plasmon resonance in the visible range of light. Generally, a powdered or crystalline form of the metal oxide is added to a solution of the platinum group metal (such as an aqueous solution). The solution of platinum group metal may be acidified (for example using hydrochloric acid or other acid) to increase the solubility of the platinum metal. Generally, the platinum metal is present in the form of a salt, for example a chloride salt (such as palladium chloride, which can be prepared by dissolving palladium chloride powder in hydrochloric acid, followed by sonication and/or stirring in a water bath). Light is then used to irradiate the solution containing the metal oxide and the platinum group metal. Generally this is achieved with UV light. It is thought that the UV light changes the valence of the platinum metal to zero (for example, palladium 2 to palladium 0) such that the platinum metal is then deposited on the metal oxide. The platinum metal is deposited on the metal oxide in the form of amorphous nanoparticles.

In some embodiments, photodeposition (for example UV photodeposition) of the platinum group metal by irradiation is carried out for less than about 60 minutes, less than about 50 minutes, less than about 40 minutes, less than about 30 minutes, less than about 20 minutes or less than about 10 minutes. In some embodiments the photodeposition is carried out for less than about 30 minutes.

Alternatively, the platinum group metal can be deposited onto the metal oxide via a photocatalytic mechanism or from nanoparticle formation in solution followed by adsorption onto the surface of the metal oxide. Preferably, the nanoparticles are deposited in an amorphous form on the metal oxide. Optionally, the method comprises the further steps of washing and/or drying the photocatalyst. The process for the preparation of the photocatalysts of the invention may further comprise a step of doping the photocatalyst. The metal oxide may be doped prior to or after mixing with the platinum group metal solution, although generally before mixing with the platinum group metal solution. In particular, the metal oxide may be doped by introducing deliberate impurities during the production of the metal oxide, such that the method of photocatalyst production is carried out on a pre-doped metal oxide.

There is also provided a photocatalyst of the invention preparable by the process described herein.

In a fifth aspect of the invention there is provided a method of purifying (or sterilising or decontaminating) a liquid or gas comprising adding a photocatalyst of the liquid or gas and exposing the liquid or gas to light in the visible range. The liquid may be water, or the gas may be air. The liquid or gas may be exposed to the light for as long as is required to purify the liquid or gas to a satisfactory degree. For example, the water may be exposed to the light for at least about 1 minute, at least about 5 minutes, at least about 10 minutes, at least about 30 minutes, at least about 60 minutes or at least about 120 minutes. The liquid may be purified to the extent that the amount of contaminants is reduced by at least about 20%, at least about 30%, at least about 40%, at least about 50%, at least about 60%, at least about 70%, at least about 80%, at least about 90%, at least about 95%, at least about 98%, at least about 99% or about 100%. The contaminants that are removed may include organic molecules and/or dyes. The purification, sterilisation or decontamination process may take place in a purification, sterilisation or decontamination device of the invention.

Generally, the photocatalyst of the invention will be removed following purification. This removal can be achieved using, for example, centrifugation or distillation.

Methods of liquid or gas purification may further comprise the steps of determining the level of liquid or gas purification, and repeating the purification steps if the liquid or gas has not reached the desired level of purity.

In a sixth aspect of the invention there is provided a method of purifying a gas (for example air) by passing the gas over or through a photocatalyst of the invention. The gas may be passed over or through the photocatalyst such that the level of impurities in the gas is reduced by desired amount. A gas being purified may be recirculated such that it is exposed to the photocatalyst of the invention multiple times. The gas may be passed through a bed of the photocatalyst. Alternatively, the gas may be mixed with the photocatalyst in solution (such as aqueous solution), for example the gas may be bubbled through a solution of the photocatalyst.

The method of gas purification may further comprises the steps of determining the level of gas purification, and repeating the purification steps if the gas has not reached the desired level of purity.

There is also provided the use of a photocatalyst of the invention in the purification of a liquid (such as water) or a gas (such as air). There is further provided the use of a photocatalyst of the invention as a gas or liquid purifier or steriliser. There is also provided the use of a photocatalyst in a method of liquid or gas decontamination.

In one embodiment of the invention there is provided a photocatalyst comprising palladium amorphous nanoparticles deposited on a TiO₂ support. The nanoparticles have a surface plasmon resonance in the visible range of light. Thus the photocatalyst is capable of catalytic activity in the visible range of light (for example, between 390 to 700 nm). The photocatalysts can be used to purify water by catalysing the degradation of contaminants and/or disrupting cell membranes of microorganisms leading to lysis of the microorganism.

Preferred features of the second and subsequent aspects of the invention are as provided for the first aspect, mutatis mutandis.

The invention will now be further described by way of reference to the following Examples which are present for the purposes of reference only and are not to be construed as being limiting on the invention. In the Examples, reference is made to a number of drawings in which:

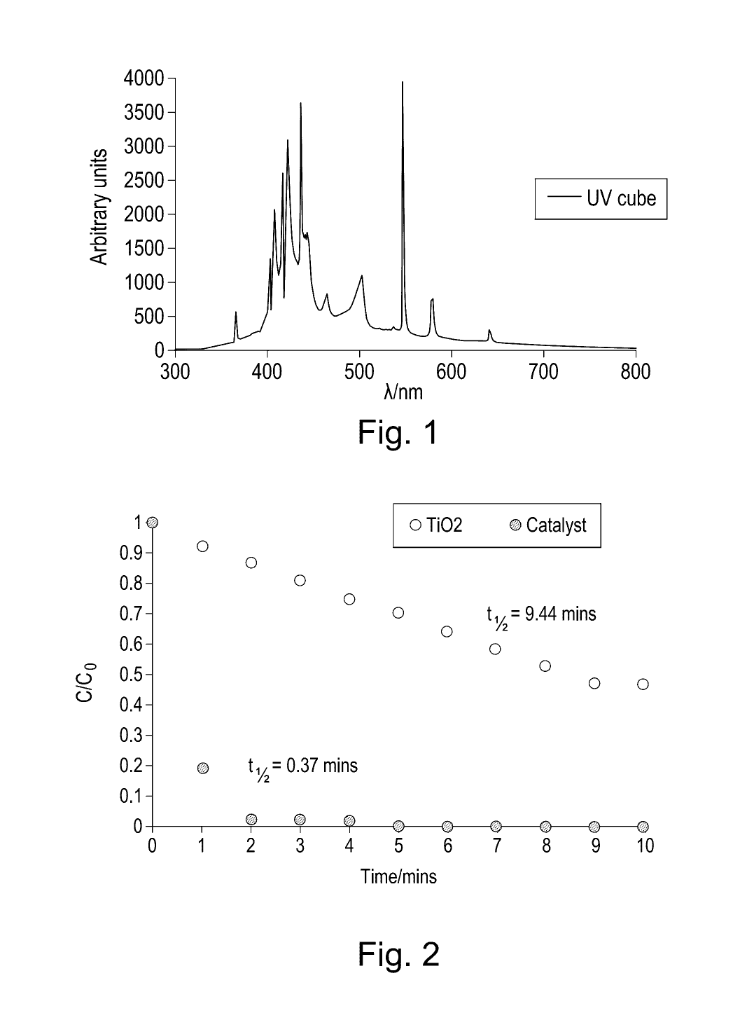

FIG. 1 shows the spectral output of a Honlé UVACUBE.

FIG. 2 shows the decolourisation of Rhodamine B by the Pd—TiO2 photocatalyst under solar conditions.

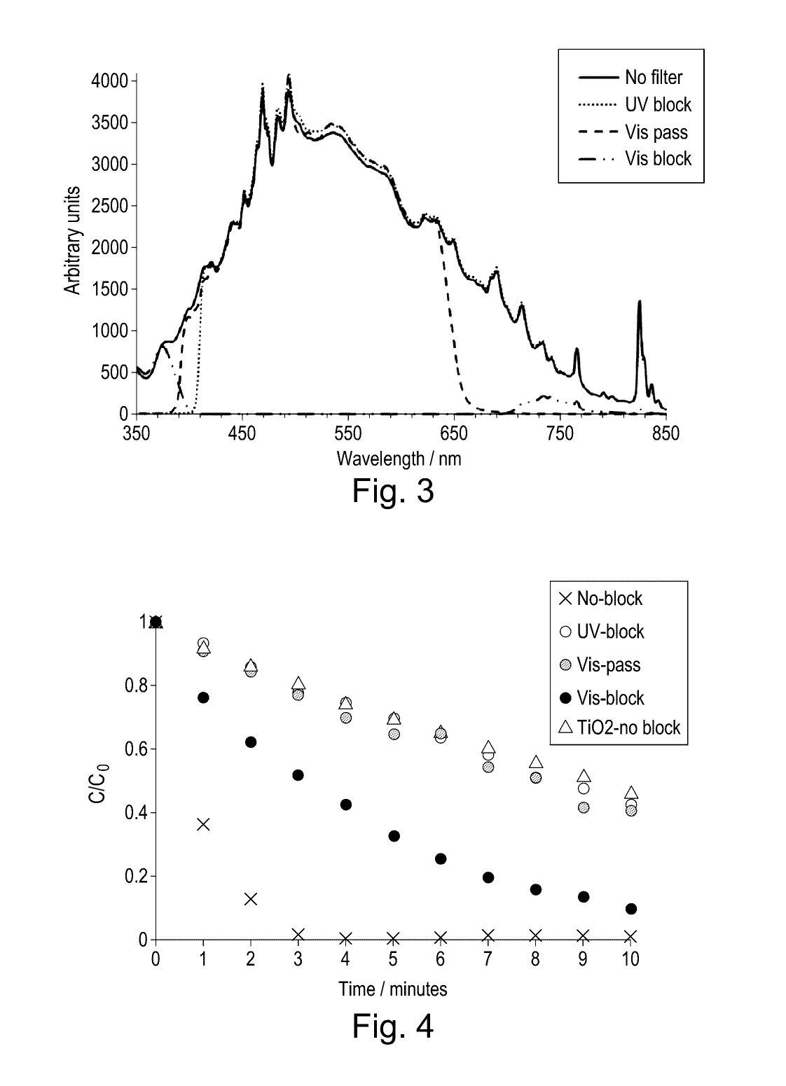

FIG. 3 shows the irradiation spectrum of the solar simulator with different filters.

FIG. 4 shows the decolourisation rates of the catalyst under different filters compared with TiO₂ under solar conditions.

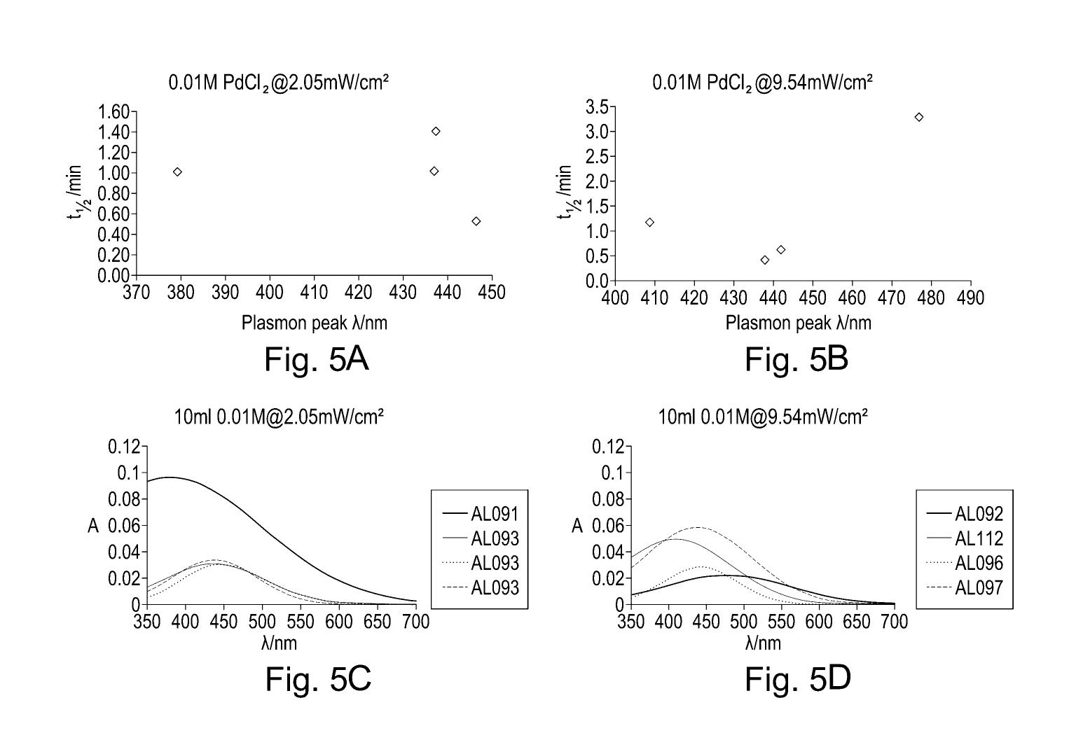

FIG. 5 shows the half-life of dye degradation versus plasmon peak position and the modelled plasmon absorption.

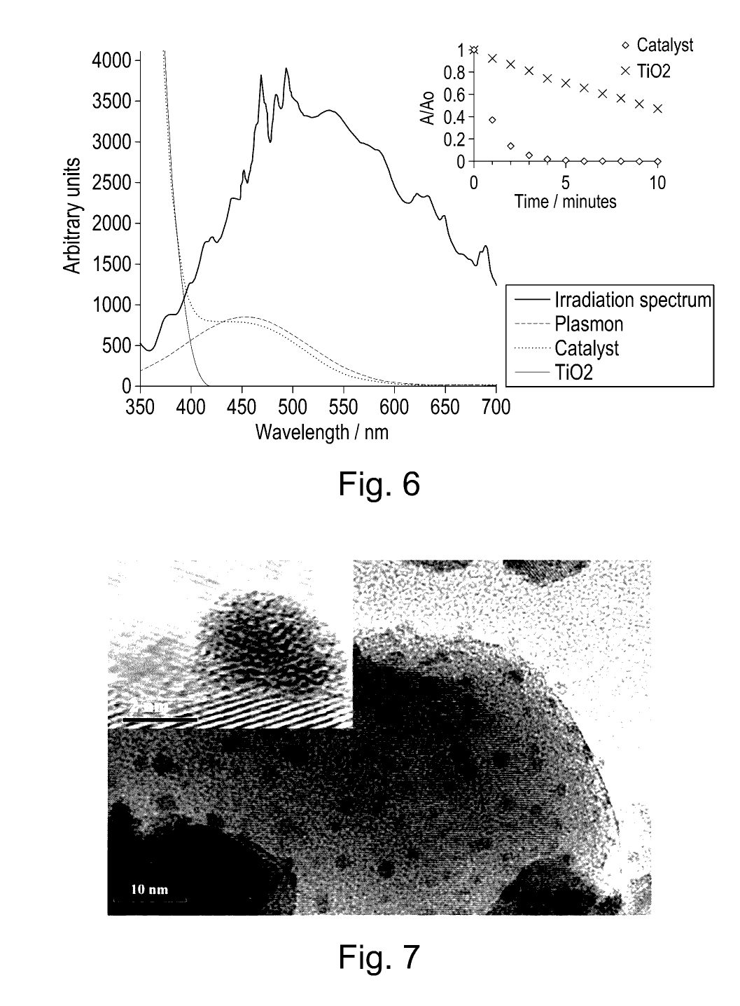

FIG. 6 shows the cut-off points for the different filters used (6a) and the decolourisation rates of the catalyst using different filters

FIG. 7 shows the TEM micrograph of the Pd deposited on TiO₂.

### EXAMPLES

### Example 1

### Photocatalysts Synthesis Procedure

Different photocatalysts were prepared using the following protocol as shown in Table 1.

TABLE 1

| | | | | | |
|---|---|---|---|---|---|
| | | | | irradiation | Plasmon |
| | Pd per gram of | dye | t_{1/2}/ | time/ | peak/ |
| Catalyst | catalyst/mg | adsorption/% | min | min | nm |

10 ml 0.01M PdCl₂ @ 2.05 mW/cm²

| | | | | | |
|---|---|---|---|---|---|
| AL094 | 5.331 | 18.3 | 0.53 | 1 | 446 |

10 ml 0.01M PdCl₂ @ 9.54 mW/cm²

| | | | | | |
|---|---|---|---|---|---|
| AL096 | 5.451 | 20.5 | 0.63 | 1 | 442 |
| AL097 | 4.851 | 10.1 | 0.43 | 0.167 | 438 |

5 ml 0.02M PdCl₂ @ 9.54 mW/cm²

| | | | | | |
|---|---|---|---|---|---|
| AL098 | 5.817 | 11.4 | 0.66 | 30 | 453 |
| AL099 | 5.924 | 18.6 | 0.55 | 3 | 438 |

5 ml 0.02M PdCl₂ @ 2.05 mW/cm²

| | | | | | |
|---|---|---|---|---|---|
| AL103 | 5.886 | 15.1 | 0.58 | 3 | 448 |

10 ml 0.02M PdCl₂ @ 9.54 mW/cm²

| | | | | | |
|---|---|---|---|---|---|
| AL106 | 6.118 | 14.2 | 0.46 | 30 | 454 |
| AL108 | 5.845 | 12.3 | 0.38 | 0.167 | 439 |

10 ml 0.02M PdCl₂ @ 2.05 mW/cm²

| | | | | | |
|---|---|---|---|---|---|
| AL109 | 6.272 | 13.9 | 0.36 | 30 | 454 |
| AL114 | 6.46 | 10.9 | 0.46 | 3 | 447 |
| AL110 | 5.407 | 14.6 | 0.52 | 1 | 428 |
| AL111 | 6.281 | 22.2 | 0.53 | 0.167 | 425 |

Stock Solution Preparation

The palladium chloride (PdCl₂) stock solution from which the Pd metal is reduced onto the titanium dioxide (TiO₂) is prepared by dissolving 177.326mg of PdCl₂ powder (for a 0.01 M solution) in 100 ml of 0.01M hydrogen chloride (HCl). First the powder and solution mixture is sonicated in a sonic bath for 30 minutes then stirred with a magnetic stir bar until the PdCl₂ is completely dissolved.

Photoreduction Procedure

For each catalyst synthesis the type of TiO₂ used is Degussa P25 nanopowder with an average particle size of 25 nm. The amount used per reaction is fixed at 1 gram.

The reaction vessel consists of a 50 mm diameter (10 mm deep) glass Petri dish containing a magnetic stir bar and sealed with a 50 mm×50 mm x lmm quartz lid to minimise evaporation during the procedure. 10 ml of PdCl₂ solution at either 0.01 M or 0.02M is used and mixed with the TiO2 for 1 minute prior to irradiation. The slurry is continuously stirred throughout irradiation during each photoreduction.

The irradiation source used is a Honle UVACUBE with a spectral output as shown in FIG. 1. Two irradiance values are used for the synthesis and these are altered by changing the distance between the irradiation source and the top of the solution inside the reaction vessel. The minimum value is 2.05 mWcm-2 and the maximum is 9.54 mWcm-2. Irradiation times are 30 minutes, 3 minutes, 1 minute, 10 seconds and 1 second.

Washing Procedure

After irradiation the slurry is transferred to a glass vial using a pipette and stored in the dark for 24 hours to allow the powder to settle. After this time the powder and solution are separated by pipette and the powder is allowed to air dry at room temperature. Once the powder is dry it is transferred to a filter system thoroughly washed with deionised water, up to 250 ml, on a paper filter base that allows the water to run through. The catalyst is then left to air dry again. When the powder is dry it is loosened with a pestle and mortar and stored in a sealed glass vial.

### Example 2

### Rhodamine B Degradation

The decolourisation of Rhodamine B (RhB) was carried out using a 50 ml solution at a concentration of 10 ppm (FIG. 2). 100 mg of the Pd—TiO2 catalyst was added to the solution and the mixture was stirred in the dark using a magnetic stir bar for 30 minutes to allow for adsorption-desorption equilibrium. The mixture was then irradiated under simulated solar condition at AM 1.5 and aliquots were taken at predetermined time intervals and centrifuged at 4000 rpm for 30 minutes to separate catalyst from solution. The solutions were then subjected to UV-vis analysis to determine the decolourisation rate. The rate of decolourisation was determined from the Langmuir-Hinshelwood model:

$$r = - \frac{d {C}_{A}}{d t}$$

where r is the rate of decolourisation, CA, is the concentration of solution and t is the time of irradiation.

An experiment was carried out to test the catalyst under more specific regions of the EM spectrum by using optical cut-off filters. Filters used were a UV light blocking filter (UV-block), a visible light blocking filter (vis-block) and a visible light pass filter (vis-pass). The Pd—TiO2 catalyst was used to decolourise RhB dye in 4 separate experiments under different irradiation conditions for each. The results of the experiments were compared with the decolourisation of TiO₂ under solar conditions without any filters. FIG. 3 shows the irradiation spectrum of the solar simulator with each of the filters attached. FIG. 4 shows the results of the 4 experiments. Decolourisation of dye by the Pd—TiO₂ without filter showed the most activity of any of the other experiments. The UV-block and vis-pass filters yielded similar results and were the least active of the experiments, which were comparable to the rate of decolourisation of dye in the presence of just TiO₂ under solar conditions without filter. The vis-block filter yielded an intermediate rate. Since TiO₂ is deactivated in the absence of UV, this suggests that it is the plasmon that is responsible for the absorption in the visible range. From the plasmon modelling data, it is clear that the centre of the plasmon sits at a region where the broad peak extends into the UV, as well as the visible region. This is further confirmed by the vis-block experimental data where the decolourisation rate increases, which can be attributed to the activation of both the TiO₂ absorption and plasmon absorption, but the visible portion of the plasmon absorption has been cut-off, leading to a decrease in activity relative to the no-block data.

### Example 3

### Surface Plasmon Analysis

The data presented here have been collected from experiments designed to test the activity of the catalyst by determining the half-life of decolourisation of Rhodamine B and also from measurements of the plasmon absorption peak using a UV-vis spectrophotometer. The raw data from the UV-vis analysis was used to model the plasmon based on a Gaussian function and fitted to the original data. The modelled plasmon and the measured plasmon absorption were consistently in good agreement and the model was used to obtain a value for the absorption of the resonance peak.

FIG. 5 shows the half-life of dye degradation versus plasmon peak position (a and b) and the modelled plasmon absorption (c and d). The irradiance value is clearly stated in the graph titles.

Dye decolourisation experiments using optical band-pass filters indicate that the increased absorption of the Pd—TiO₂ catalyst is due to the presence of localised SPR. This is evident when a UV cut-off filter was used to ‘deactivate’ the TiO₂ by prohibiting the incidence of super band gap photons, (FIG. 3 and FIG. 4), into the reaction vessel. Despite the presence of the cut-off filter, a significant amount of RhB decolourisation under visible light irradiation still occurred and is thought to be attributed to the plasmon. By blocking visible light irradiation, an even greater amount of dye was degraded in the same time frame relative to UV-blocking. This suggests that the plasmon is also active in the UV region, contributing to the overall degradation under these conditions. This is supported by the modelled plasmon peak showing a broad absorption extending into the UV from its central point. FIG. 7 shows the absorption of TiO2 Degussa P25 before photochemical deposition of Pd metal compared with the absorption of the Pd—TiO₂ catalyst. The modelled plasmon and the broadband irradiation spectrum used for photodegradation are also included. The inset shows the results of photodecolourisation of RhB of TiO₂ compared with the Pd—TiO₂ catalyst under simulated solar conditions.

The structure and size of the Pd nanoparticles were confirmed by TEM analysis. The micrographs reveal that the Pd nanoparticles are amorphous in nature and have a diameter of less than 5 nm as shown in FIG. 8.

1. A photocatalyst capable of catalytic activity in the visible range of light comprising amorphous platinum group metal nanoparticles on a metal oxide.

2. A photocatalyst of claim 1, wherein the nanoparticles have surface plasmon resonance in the visible range of light.

3. A photocatalyst according to claim 1 wherein the nanoparticles are deposited on the metal oxide support, optionally by UV photodeposition.

4. A photocatalyst according to claim 3 wherein the UV photodeposition is carried out for less than 30 minutes.

5. A photocatalyst according to claim 1 wherein the nanoparticles have a size of between about 2 and about 5 nm.

6. A photocatalyst according to claim 1 wherein the platinum group metal is platinum or palladium.

7. A photocatalyst according to claim 1 wherein the metal oxide is a refractory metal oxide.

8. A photocatalyst according to claim 7, wherein the refractory metal oxide is titanium, chromium, zirconium, niobium, molybdenum, hafnium or tungsten.

9. A photocatalyst according to claim 1, wherein the metal oxide is a titanium metal oxide.

10. A photocatalyst according to claim 9, wherein the titanium metal oxide is TiO₂.

11. A photocatalyst according to claim 1 wherein the metal oxide is doped.

12. A photocatalyst according to claim 11, wherein the photocatalyst is doped with nitrogen.

13. A purification device comprising a photocatalyst according to claim 1.

14. The purification device of claim 13, wherein the device comprises a reaction chamber having an inlet and an outlet, and a source of visible light, and further wherein the photocatalyst is contained within the reaction chamber.

15. The purification device of claim 14, wherein the source of visible light is external to the reaction chamber and the reaction chamber is transparent to the visible light.

16. The purification device of claim 14, wherein the reaction chamber inlet and reaction chamber outlet comprise valves for controlling the flow of liquid or gas.

17. The purification device of claim 14, wherein the purification device further comprises a storage chamber for storing purified liquid or gas, wherein the storage chamber is in fluid communication with the reaction chamber via the reaction chamber outlet, optionally wherein the storage chamber further comprises a dispensing outlet having a valve.

18. The purification device of claim 14, further comprising:
a) a pump for feeding liquid or gas into the reaction chamber via the inlet; and/or
b) a pump for expelling purified liquid or gas from the reaction chamber via the outlet.

19. The purification device of claim 17 further comprising a pump for dispensing purified liquid or gas from the storage chamber via the dispensing outlet.

20. The purification device of claim 16, further comprising control means for controlling the flow of liquid or gas through the purification device, the control means being operably linked with one or more of the valves and/or pumps.

21. The purification device of claim 14, further comprising means for removing the photocatalyst from the purified liquid or gas.

22. A hydrogen production device comprising a photocatalyst according to claim 1.

23. The hydrogen production device of claim 22, wherein the device comprises a reaction chamber having a liquid inlet, a gas outlet, and a source of visible light, and further wherein the photocatalyst is contained within the reaction chamber.

24. The hydrogen production device of claim 23, further comprising a storage chamber in fluid communication with the reaction chamber for storing liberated hydrogen, optionally wherein the storage chamber further comprises a dispensing outlet having a valve.

25. The hydrogen production device of claim 23, wherein the reaction chamber further comprises a waste outlet.

26. The hydrogen production device of claim 23, further comprising valves to control the flow liquid into the reaction chamber via the liquid, the flow of gas out of the reaction chamber via the gas outlet, and/or the flow of waste through the waste outlet.

27. The hydrogen production device of any claim 23, further comprising:
a) a pump for feeding liquid into the reaction chamber via the liquid inlet;
b) a pump for expelling liberated hydrogen gas via the gas outlet;
c) a pump for expelling waste via the waste outlet, if present; and/or
d) a pump for expelling purified gas from the storage chamber via the dispensing outlet, if present.

28. The hydrogen production device of claim 23, further comprising control means for controlling the flow of liquid or gas through the hydrogen production device, the control means being operably linked to one or more of the valves and/or pumps.

29. A process for preparing a photocatalyst capable of catalytic activity in the visible range of light comprising depositing platinum group metal nanoparticles on a metal oxide.

30. A process according to claim 29, wherein the platinum group metal nanoparticles are deposited by irradiation.

31. A process according to claim 30 wherein the irradiation is carried out by UV photodeposition and optionally the photodeposition is carried out for less than 30 minutes.

32. A process according to claim 29 wherein the platinum group metal is in solution.

33. A process according to claim 29 wherein the metal oxide is in the form of solid, optionally a powder or in crystalline form.

34. A process according to claim 33 wherein the metal oxide solid is added to a solution of the platinum group metal.

35. A process according to claim 29 wherein the photocatalyst is dried after irradiation.

36. The process according to claim 29, wherein the metal oxide is titanium dioxide.

37. A process according to claim 29, wherein the platinum group metal is palladium.

38. A method of gas or liquid purification, sterilisation or decontamination, comprising mixing the liquid or gas with a photocatalyst of claim 1, and applying visible light to the resulting mixture.

39. The method of claim 38, wherein the liquid is water.

40. The method of claim 38, wherein the gas is air.

41. A photocatalyst obtainable according to the process of claim 29.
